(19) United States
(12) Patent Application Publication
(10) Pub. No.: US 2007/0265710 A1
(43) Pub. Date: Nov. 15, 2007

(54) Method of making hernia patch and resulting product

(75) Inventors: Roderick B. Brown, Glenwood, MN (US); Michael Afremov, St. Louis Park, MN (US)

Correspondence Address: NIKOLAI & MERSEREAU, P.A., 900 SECOND AVENUE SOUTH, SUITE 820, MINNEAPOLIS, MN, 55402, US

(73) Assignee: Minnesota Medical Development, Plymouth, MN (US)

(21) Appl. No.: 11/431,171

(22) Filed: May 10, 2006

(51) Int. Cl. A61F 2/02

(52) U.S. Cl. 623/23.72

(57) ABSTRACT

A hernia patch comprising a frame made of a plurality of strands made from a shape memory alloy wound together as a cable and a prosthetic mesh material attached to the cable frame. The cable frame forms a loop of a predetermined shape when unconstrained. The cable frame cam is rolled or folded into a tight cylindrical shape and inserted into a small diameter trocar. When the hernia patch is ejected out of the trocar into the patient's abdominal cavity, the frame warms to the point where the alloy is in its austenite form so that it springs to a functional, predetermined configuration. Alternatively, the superelastic properties of the alloy cause the frame to return to the predetermined configuration. The frame is integral with the prosthetic mesh material so that it will not migrate and therefore will not need to be sutured or stapled in place.

### BACKGROUND OF THE INVENTION

I. Field of the Invention

This invention relates to an apparatus to be used in hernia repair surgery, and more particularly to a prosthetic hernia repair patch that can be rolled into a tube for laparoscopic delivery through a trocar and which deploys to a generally planar form when ejected from the trocar into the abdominal cavity.

II. Discussion of the Prior Art

Implantable mesh patches for the repair of inguinal and other abdominal wall hernias are well known in the prior art. Typically these patches are intended for permanent placement within a patient's body space. For example, my U.S. Pat. No. 5,824,082 issued on Oct. 20, 1998 for a “Patch for Endoscopic Repair of Hernias” teaches a prosthesis for use in hernia repair surgery having a preformed prosthetic fabric supported along its periphery by shape memory alloy wire having a transformation temperature corresponding to normal body temperature, allowing the prosthesis to be tightly rolled into a cylindrical configuration for delivery.

Laparoscopic surgery has proven to be a preferred surgical technique for addressing inguinal hernias. The '082 patent facilitated laparoscopic procedures by providing a hernia repair patch supported by a single strand of wire Nitinol frame. The patch could be rolled up and inserted into a cannula and then deployed through the cannula into the body to cover the direct and indirect hernia space. Because the frame of the '082 patent is integral to the patch, it does not migrate and need not be sutured or stapled in place.

It has been found, however, that smaller sized cannulas are often preferred in laparoscopic procedures. Patients find that trocars with a smaller diameter are less invasive and less painful. A need, therefore, exists for a hernia patch to be used in laparoscopic surgery that is prefabricated to conform to anatomical structures, that readily deploys when released from a tubular laparoscopic introducer, that will remain in place without a need for stapling or suturing to the underlying fascia, and which is flexible enough to be rolled or folded to fit into a trocar of a smaller diameter. The present invention fulfills that need.

### SUMMARY OF THE INVENTION

The hernia repair patch of the present invention includes a frame comprising a plurality of fine strands of a suitable shape memory alloy wound together as a cable. The cable frame forms a loop of a predetermined shape when unconstrained. A synthetic prosthetic material, such as polytetrafluorethylene, or a polypropylene mesh is attached to and supported by the cable frame. The cable frame supporting the mesh material may be formed from Nitinol or a suitable shape memory polymer, such as polynorbornen, and can be attached to the prosthetic material so that it has a somewhat hourglass shape when the shape memory material is in its austenite form and a rolled, cylindrical shape or a folded configuration when in a martensite form. Because the frame is a cable of a plurality of strands, the rolled, cylindrical shape or folded configuration can be tighter and fit in smaller diameter trocars.

In accordance with one embodiment, the atomic percent of nickel in the Nitinol alloy is such that the alloy exhibits a transformation temperature at about 37 degrees Celsius (body temperature). Polynorbornen exhibits a similar transformation at body temperature. Thus, when the patch is cooled, it can be readily formed into a cylindrical configuration for placement in the delivery trocar. When ejected out of the trocar into the patient's abdominal cavity, the frame warms to the point where the alloy is in its austenite form so that it springs into a functional, predetermined configuration.

Alternatively, rather than depending on the temperature responsive properties of the alloy, advantage can be taken of the superelastic properties of the alloy. Here, stress induced martensite is achieved during rolling or folding of the frame. Upon release from the confines of the cannula, the frame rebounds to its preformed shape. The narrowed central portion of an hourglass shape patch accommodates the inferior epigastric vessels and cord structures while the opposed lobes will cover the direct and indirect hernia space. The frame is integral to the patch such that it will not migrate and therefore will not need to be sutured or stapled in place.

The foregoing features, objects and advantages of the invention will become apparent to those skilled in the art from the following detailed description of the preferred embodiment, especially when considered in conjunction with the accompanying drawings.

### DESCRIPTION OF THE DRAWINGS

FIG. 1 is an enlarged plan view of the hernia repair patch constructed in accordance with the present invention;

FIG. 2 shows the patch of FIG. 1 in a rolled or folded, tubular configuration of endoscopic delivery through a trocar;

FIG. 3 is a greatly enlarged cross-section taken through cable frame 12 in FIG. 1;

FIG. 4 is a cross-section view of the junction of opposed ends of the cable frame used in the hernia patch of FIG. 1;

FIG. 5 is a perspective view of the heat set mold used to form the cable frame of the present invention; and

FIG. 6 is a second embodiment of the present invention wherein the patch is adapted to cover an abdominal hernia.

### DESCRIPTION OF THE PREFERRED EMBODIMENT

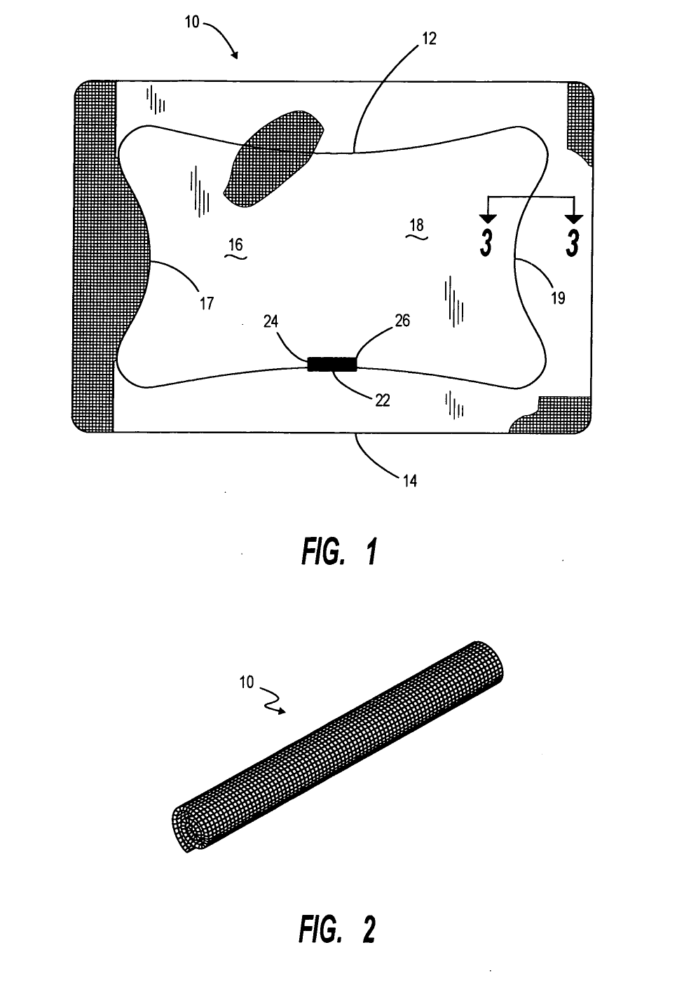

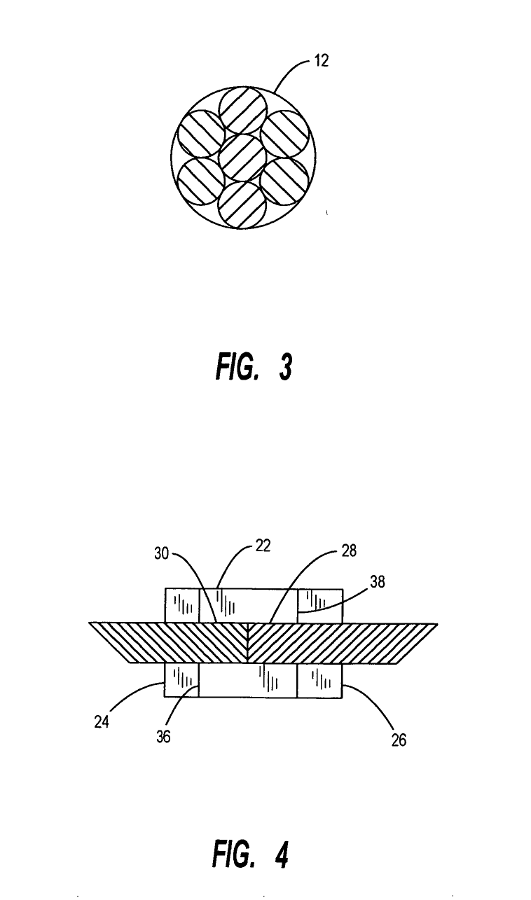

Referring to FIG. 1, there is illustrated an enlarged view of the hernia repair patch constructed in accordance with the present invention. In this embodiment, the patch is sized to cover an inguinal hernia. The patch prosthesis is indicated generally by numeral 10 and includes a frame member 12 supporting a mesh fabric sheet 14. The frame 12 comprises a plurality of metal wire or plastic strands wound together to form a cable as shown in the cross-sectional view of FIG. 2. The cable forms a loop of a predetermined shape configuration when unconstrained. In the preferred embodiment, the loop has a somewhat hourglass or dog bone shape with a first and second lobe section 16 and 18. At each end of the hourglass shaped loop, the frame has a first and second concaved section 17 and 19. The ends of the cable 24 and 26 are joined together in a metal (preferably Nitinol) ferrule member 22. The ends of the cable may be laser welded to the ferrule or may be crimped within the ferrule, thus forming an endless loop.

The plurality of wire strands are also preferably made from a shape memory alloy, such as Nitinol. Nitinol is the preferred shape memory alloy because it is both commercially available and well known to be useful in medical prostheses. Because Nitinol wires are characterized as being super elastic or “pseudo elastic”, it helps the frame in being rolled up or folded so that the patch may be inserted into a lumen of a small diameter cannula and returned to its expanded configuration (shown in FIG. 1) after deployment. Because the frame is made from a cable of Nitinol wire strands, as opposed to a single wire strand as in the '082 patent, the frame has a greater degree of flexibility so that it may be rolled or folded into a smaller, more compact form for insertion in to the lumen of a cannula having a reduced diameter compared to what is needed when a single wire is used for the frame. Once the cable frame is deployed by pushing it from the distal end of the cannula, the plurality of Nitinol wire strands will transition from the martensite form to the austenite which may be made to occur at body temperature or at some other temperature. Instead of using metal, shape memory alloy strands in the cable, it can comprise plural fine strands of suitable shape memory polymer, with polynorbornen being preferred.

Supported by the cable frame 12 is the prosthetic mesh fabric 14. The mesh fabric 14 is preferably woven strands of a polypropylene plastic or expanded PTFE (Gortex). Of course, any fabric that is body compatible and capable of being steam sterilized, or is a monofilament material resistant to infection may be used. Therefore, any material used in prior art hernia patches would also be acceptable substitutes for the mesh fabric. The mesh fabric is preferably attached to the frame 12 by stitching the fabric to the frame, but thermal bonding is also an option as is integration into the mesh or fabric at the time of manufacture. As shown in FIG. 1, the mesh fabric 14 extends beyond the perimeter of the frame.

In use, lobe members 16 and 18 are adapted to be positioned over the direct and indirect hernia spaces. When the frame member 12 is stressed or cooled below the transformation state of the shape memory alloy so that it is in its martensite form, the prosthesis 10 can be tightly helically wrapped or folded to form a cylindrical helical structure as illustrated in FIG. 2. This allows the prosthesis to be introduced into the abdominal cavity through a tubular cannula. As the shape memory alloy frame 12 warms to body temperature, or is expelled from the lumen of the cannula the frame transforms from its martensite state to its austenite form as depicted in FIG. 1. Using a laparoscopic forceps, the prosthesis 10 of FIG. 1 can be grasped and positioned by a surgeon until lobes 16 and 18 are appropriately located for covering the hernia defect but without interfering with the other anatomical structures.

To form the cable 14 to a desired shape when unconstrained, multiple strands of Nitinol wire are wound together forming cable whose opposed ends are joined together to from a closed loop. Preferably, and as shown in FIG. 4, the ends are closed by inserting the ends 24, 26 into a tubular ferrule 22 and the ferrule 22 is then laser welded to the cable ends.

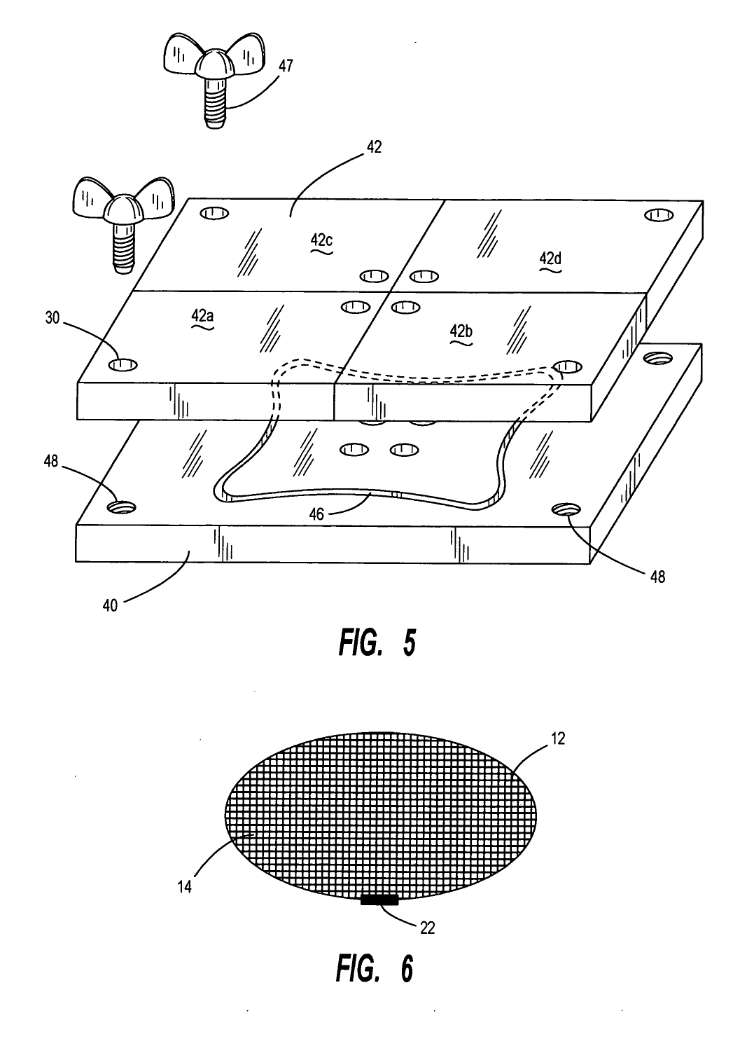

FIG. 5 shows a mold structure for use in establishing the desired frame shape to the cable loop. It comprises a base plate 40 having a recess 46 formed therein. The recess 46 defines the desired shape configuration for the frame 14. The cable 14 is then fitted piecewise into the recess 46 so that the cable follows the perimeter of the recess. The metal cover plate 42 comprising segments 42a-42d is then affixed to the base plate 40 in pieces as the cable is being forced into the recess 46 to prevent the cable 14 from escaping the recess 46. The segments comprising the cover plate 42 is then secured to the base plate 40 by passing fasteners 47 through cooperating threaded bores 30, 48 in the cover plate 42 and base plate 40, respectively. The cover plate is preferably formed from plural segments, thereby allowing piece wise insertion of the cable 14 into the groove or recess 46.

Once inserted, the assembly is then subjected to a heating step for a time and at a temperature that imparts a set to the closed loop. After the assembly is sufficiently cooled, the top plate segments 42a-42d are unscrewed and cable 14 is removed from the recess 46. The mesh fabric 14 is affixed to the closed loop 12.

A second embodiment of the present invention used to address abdominal hernias is shown in FIG. 6. In this embodiment, the frame 12 is generally oval-shaped, and the mesh fabric 14 is co-extensive with the frame 12.

In use, the hernia patch 10 is deployed in much the same way as the hernia patch of my '082 patent and that method is hereby incorporated by reference.

While the invention has been shown in one of its forms, it should be apparent that it is not limited to these embodiments, but is susceptible to various changes without departing from the scope of the invention.

What is claimed is:

1. A hernia patch for laparoscopic delivery comprising:
(a) a frame member comprising a plurality of strands of a material exhibiting a shape memory property wound together as a cable and forming loop of a predetermined shape configuration when unconstrained;
(b) a mesh fabric attached to the frame member and arranged to be rolled up or folded for insertion through a tubular cannula into an abdominal space and when ejected from the cannula will assume the predetermined shape configuration.

2. The hernia patch as in claim 2 wherein the mesh fabric extends beyond a perimeter of said loop.

3. The hernia patch as in claim 2 wherein the mesh fabric is attached to the frame member by stitching.

4. The hernia patch as in claim 2 wherein the mesh fabric is polypropylene.

5. The hernia patch as in claim 1 wherein the mesh fabric is polytetrafluorethylene.

6. The hernia patch as in claim 1 wherein the strands are a Nitinol alloy.

7. The hernia patch as in claim 1 wherein the strands are a shape memory polymer.

8. The hernia patch as in claim 1 wherein the shape memory polymer is polynorbornen.

9. The hernia patch as in claim 6 wherein the loop is an endless loop formed by joining the opposed ends of said cable in a ferrule formed from Nitinol alloy.

10. The hernia patch as in claim 1 wherein the cable is formed from Nitinol strands, each at least 0.0005 in. diameter.

11. The hernia patch as in claim 10 wherein the cable comprises from at least two strands.

12. The hernia patch as in claim 1 wherein the predetermined shape configuration is oval.

13. The hernia patch as in claim 12 wherein the mesh fabric is co-extensive with the frame member.

14. The hernia patch as in claim 1 wherein the predetermined shape configuration comprises a generally trapezoidal shape having concave sides meeting at generally convex vertices.

15. A method of making a hernia patch comprising the steps of:
(a) forming a cable comprising multiple strands of a shape memory material into a closed loop by joining opposed ends of the cable together;
(b) providing a metal base plate having a recess formed therein defining a desired shape configuration for a hernia patch frame when the frame is unconstrained;
(c) fitting the closed loop into said recess;
(d) affixing a metal cover plate to the base plate for retaining the closed loop in the recess;
(e) heating the assembly of step (d) for a time and at a temperature for imparting a set to the closed loop;
(f) cooling the assembly following step (e); and
(g) affixing the mesh fabric to the closed loop of step (f).

16. The method of claim 15 wherein the opposed ends of the cable are joined by:
(a) inserting the opposed ends into a tubular ferrule; and
(b) welding the ferrule to the opposed ends of the cable.

17. The method as in claim 15 wherein the mesh fabric is affixed to the closed loop by sewing stitches.

18. The method as in claim 15 wherein the mesh fabric is affixed to the closed loop by one of adhesive, ultrasonic thermal bonding.

19. The method as in claim 15 wherein the desired shape configuration is an oval.

20. The method as in claim 15 wherein the desired shape configuration is generally a dog-bone shape configuration.

21. The method as in claim 15 wherein the desired shape configuration is generally trapezoidal with concave sides meeting at generally convex vertices.

22. The method as in claim 15 wherein the frame is interwoven into the mesh fabric.

23. The method as in any one of claims 15-22 wherein the shape memory material is Nitinol.

24. The method as in any one of claims 15-22 wherein the shape memory material is a polymer.

25. The method as in claim 24 wherein the polymer is polynorbornen.
